(19) United States
(12) Patent Application Publication
(10) Pub. No.: US 2009/0185951 A1
(43) Pub. Date: Jul. 23, 2009

(54) Volatile Liquid Disseminating Device

(75) Inventors: Colin Litten-Brown, Bracknell Berkshire (GB); Guy Edward Naish, Bicester Oxfordshire (GB); Kishen Gohil, Surrey (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 12/299,504

(22) PCT Filed: Apr. 27, 2007
(86) PCT No.: PCT/CH2007/000207

§ 371 Date: Nov. 21, 2008

(30) Foreign Application Priority Data
May 4, 2006 (GB) 0608774.6

(51) Int. Cl. A61L 9/12

(52) U.S. Cl. 422/5; 261/18.1

(57) ABSTRACT

A device adapted to disseminate into an atmosphere in a desired sequence and timing, a plurality of active volatile liquids, such as fragrances, comprising a plurality of individual liquid reservoirs, a dissemination element associated with each reservoir, a source of forced air current and a conduit from the source of forced air current to each dissemination element, the conduit being a single conduit that is adapted to be movable so as to supply air current to each dissemination element in a desired sequence.

The device may be provided as a simple plug-in type and overcomes the problem of “habituation” encountered with a single fragrance.

This invention relates to devices for disseminating a plurality of volatile liquids into an atmosphere.

Devices for disseminating into an atmosphere an active volatile liquid are well known to the art. By “active” is meant a liquid whose presence in the atmosphere is desired for the performance of some function—typical examples include a fragrance, a disinfectant, an odour masking agent or a fungicide. A wide variety of such devices is commercially available. While most of these rely on evaporation alone, many have assisted dissemination, in that the evaporation is augmented by a heating element, a forced air current or both. These are generally powered by electricity, supplied by internal or external batteries, solar cells or mains power. One common and particularly desirable type of augmented evaporation device is the so-called “plug-in” type, a relatively small device that comprises, in a single unit, reservoir, dissemination element (such as a porous wick), electrically-driven augmentation element (such as heater and/or fan), transformer and power outlet pins, allowing it to be plugged directly into a power outlet. Most of these disseminate only one liquid.

It is advantageous to be able to disseminate two or more different liquids sequentially. For example, in the field of air fresheners, it is known that people become accustomed to a single fragrance (the art describes this as “habituated”) and it loses its effectiveness. Dissemination of a second, different fragrance after a suitable time can counter this. It may also be useful to disseminate entirely different liquids, for example, a fungicide, followed by a fragrance to mask the odour of the fungicide.

The problem of multiple liquids has been addressed by the art. One means is to provide a plurality of volatile materials on a rotatable plate or cartridge, in which the desired liquid is rotated into a dissemination position, that is, a position in which it is brought into juxtaposition with a disseminating means, such as a heat source or a forced current of air from a fan or an impeller. Typical examples of such devices are described in US published applications 2002/0068010 and 2004/0009103. While undoubtedly effective, such devices are large and mechanically complex, one drawback of which is that they can never be available as plug-in devices. Moreover, it is necessary to buy individual cartridges, with a restricted choice of fragrances and a quantity restricted by the size and nature of the cartridge.

Another proposed means is to provide stationary fragrances and direct an air current to the desired fragrance alone. This generally involves having a multiplicity of pathways, one per liquid, and blocking the pathways to all but the desired liquid. Typical examples include PCT published applications WO2003/028775 and WO2004/096300 and International applications PCT/CH2006/000011 and PCT/CH2006/000142. In the first two of these documents, the devices described are, by their nature, limited to two liquids. In the case of second two, this problem is overcome, but the devices are relatively bulky and not suitable for a small plug-in device.

It has now been found that it is possible to provide, in a compact, simple, inexpensive device, a plurality of volatile liquids that can be disseminated into an atmosphere in a regulated fashion. The device may be provided as a plug-in device. The invention therefore provides a device adapted to disseminate into an atmosphere in a desired sequence and timing, a plurality of active volatile liquids, comprising a plurality of individual liquid reservoirs, a dissemination element associated with each reservoir, a source of forced air current and a conduit from the source of forced air current to each dissemination element, the conduit being a single conduit that is adapted to be movable so as to supply air current to each dissemination element in a desired sequence.

The invention further provides a method of providing sequentially in an atmosphere a plurality of volatile liquids, comprising the steps of

(a) providing each of a plurality of volatile liquids in individual reservoirs with dissemination elements extending therefrom; and

(b) providing to the dissemination elements in a desired order and timing a forced air current from a conduit, which conduit is movable so as to direct air current to the desired dissemination element.

The device hereinabove mentioned comprises four essential elements:
a plurality of liquid reservoirs;
a dissemination element associated with each reservoir;
a source of forced air current; and
a conduit able to be moved to each dissemination element as required.

The reservoir can be any suitable reservoir. It may be made of any suitable material or of any suitable size, shape or configuration, and the skilled person can easily determine suitable characteristics in each individual case. Typical materials include glass, metals, plastics, suitably lined paper and cardboard and ceramics. In one particular embodiment, the reservoir is made of a transparent or translucent material so that the liquid level can be seen, thus making it easy to determine when the liquid has been exhausted and the reservoir requires refilling or replacing. One of the advantages over the prior art is that, consistent with the number of reservoirs and overall desired size of the device, the reservoirs can be of a relatively large capacity in comparison with the capacities of the cartridges of the prior art. Moreover, they permit of a more versatile apparatus, as the user is no longer constrained to the liquids on a particular cartridge—the user can choose new liquids at any time. The reservoirs can be made available as refills for easy insertion into an existing apparatus. A typical refill will have the shape of a bottle of any suitable cross-section.

The dissemination element may be any element suitable for the conveyance of liquid from a reservoir to an atmosphere, at which point dissemination takes place because of an airflow impinging on the element. A typical dissemination element is a porous wick, of the type well known to the art and widely used in commercial air fresheners. Such wicks may be made of any suitable material, for example, porous plastics, compressed cellulosic material, dried plant materials, and sintered powdered metals or ceramics. The wicks may be cylindrical or they may be stamped out of a flat, porous material, such as porous cardboard. Another type of dissemination element is an external capillary emanator, that is, a surface or solid on the surface of which channels of capillary dimensions have been formed. This type of element has the advantage of avoiding the “fractionating” effect that some wick materials can have on fragrances that are complex mixtures. A typical example of a capillary emanator is described in U.S. Pat. No. 4,913,350.

The source of forced air current may be any suitable source. It may be, for example, a compressed gas stored in a bottle or aerosol and gradually released. It may also be a fan or impeller, which rotates and forces surrounding air to flow. The electrical power to drive such a fan may be supplied by any convenient means, such as batteries, solar cells and mains electricity. In the case of mains electricity, the device can comprise power pins for direct plugging into a power point and a transformer to reduce the mains voltage to a suitable level. The fans or impellers used are typically the small blowers used to cool laptop computers, and they are readily commercially available in a wide variety of sizes and types.

The conduit that carries the air current to the dissemination elements is a single conduit. That is to say, it is a single, essentially tubular member of any desired cross-sectional area and shape, which can be moved to convey air current to any selected dissemination element. This means that the conduit must be capable of being directed to convey air to any desired dissemination element, which means in turn that the conduit or a part thereof must be able to move or bend. In both cases, while straight-line lateral movement, movement in three dimensions and combinations thereof are possible, it is simpler and cheaper to arrange pivoting movement of the conduit or part thereof about a point, such that the end of the conduit remote from the pivot point moves in a single plane in an arc. On this arc are located the dissemination elements through which the volatile liquids are conveyed from their reservoirs. The construction of a device will from this point be described in terms of such a device, which is especially useful for small plug-in devices, but the invention is not limited thereto, and more complex devices utilising more complex modes of operation are also included.

The conduit or part thereof may either be made of a flexible material or it may be rigid and pivotable. There are many ways of achieving this and the skilled person will readily be able to envisage a particular method suitable for any given desired construction of device. One particular construction comprises a rigid, tubular conduit that extends from a pivotable housing partially or completely surrounding a source of the forced air current, typically a blower rotating in the same plane as the plane of rotation of the housing. Thus, as the housing pivots, the end of the conduit remote from the housing (the “exhaust end”) swings through an arc and the conduit may be directed at any one of the disseminating elements located along this arc. In a variation on this construction, the pivotable member is not the housing but part of the conduit itself, so that the housing and the initial part of the conduit leading therefrom remain stationary and the remainder of the conduit pivots. The mechanical construction of such pivotable air current-conveying conduits is well known to the art.

The orientation of the conduit is not critical for the working of the invention. It can, for example, move in a horizontal plane. However, for a more compact device, such as a plug-in device, a conduit moving in a vertical plane may be used. This has the advantage with some embodiments (as will be hereinafter described in more detail with reference to a particular embodiment) that gravity may be used to assist the moving of the conduit. Naturally, if desired, a conduit may be made to move in a plane that lies between horizontal and vertical, or it may be movable in three dimensions rather than two. Such lie within the skill of the art, but they are naturally more complex and usually more bulky, and are preferred only for more sophisticated uses.

The means for directing the exhaust end of the conduit to any desired disseminating means (“directing means”) may be any such means known to the art. Again, many such directing means are known and available to the art, and the precise nature of the means in each case may be suited to the particular nature of the conduit. Typical examples of suitable directing means include solenoids acting either directly or through mechanical linkages, motors with a rack and pinion or worm gear linear drive, hydraulic or pneumatic pressure lines and electromagnets. In one particular embodiment, a series of electromagnets is positioned such that, when any one electromagnet is switched on, it attracts the movement of the conduit by means of a magnet or a ferromagnetic material located on the conduit, such that the forced air current blows from the exhaust end of the conduit over an adjacent disseminating means. In a variation on this electromagnetic directing means, the conduit exhaust end swings in an arc in a vertical plane, and a series of electromagnets positioned appropriately along the arc stop the conduit in a liquid disseminating position. In such an arrangement, the number of reservoirs and disseminating elements is generally an odd one, meaning that the middle disseminating element is located vertically below the pivot point. Thus, as an electromagnet retaining the conduit in liquid disseminating position is switched off, its neighbouring electromagnet is switched on, and the conduit moves into a new position. When the conduit moves in a vertical arc, gravity will assist the movement downwards and help the conduit to locate at the vertical position. However, it is also possible to have even numbers of reservoirs.

In the embodiment previously described, typical numbers of reservoirs are three and five. These allow for a good compromise of simplicity, compact dimensions and reasonable capacity of each reservoir. There may be more, but one or more of these features is compromised to some extent.

The order in which the reservoirs and associated dissemination elements receive the forced air current and the duration of that exposure can be regulated automatically by known means. For example, the device may be programmable, such that a preselected dissemination of different liquids may be achieved. In the case of the vertically-arranged electromagnet devices mentioned hereinabove, each electromagnet may be actuated in turn and for a desired time. However, it is not necessary that the exhaust end of the conduit move from one dissemination element to its immediate neighbour. Depending on the nature of the device, it may be possible to select any desired sequence. Such sophistication may not be necessary in all cases, for example, in a relatively simple plug-in device, but the invention allows it when it is necessary.

The devices of this invention may be made economically from known materials and by known techniques. For example, in the case of simple devices such as plug-in air fresheners, only a few special plastics mouldings may be necessary, the rest being made from standard off-the-shelf components. The simpler plug-in devices of the invention offer for the first time the sophistication of a multi-liquid device in a simple, reliable, compact apparatus. Such devices may be easily refillable and be programmed to disseminate their liquids in any desired order and for any desired length of time.

The invention is now further described with reference to the drawings, which depict a preferred embodiment and which are not intended to be in any way limiting.

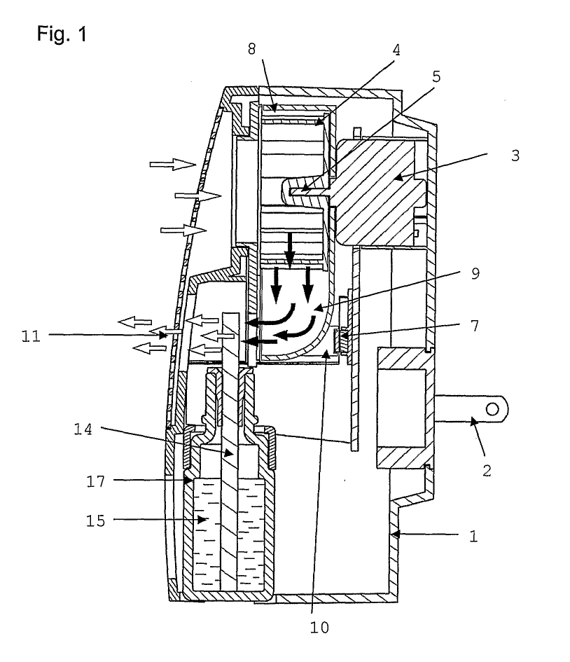

FIG. 1 is a vertical front-to-back cross-section of a device.

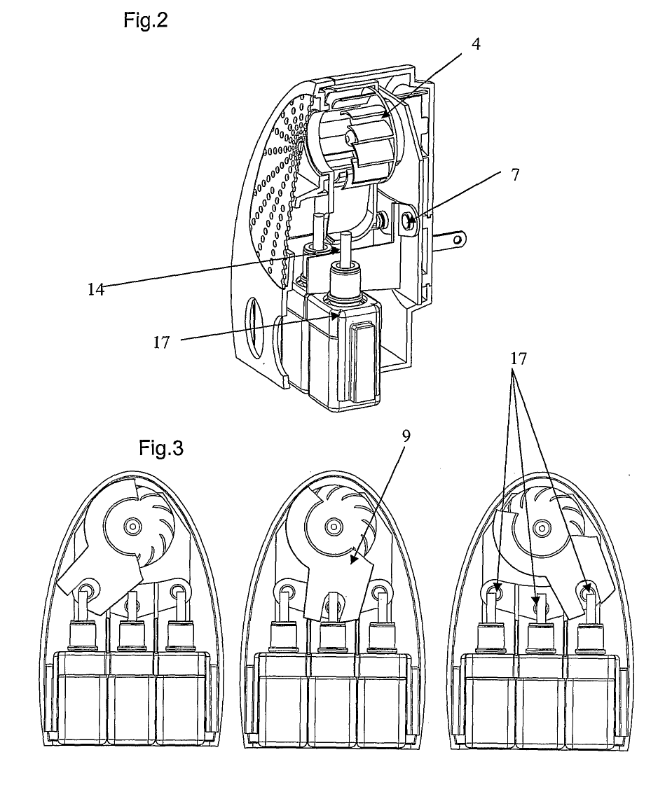

FIG. 2 is a perspective part-sectional view of the device of FIG. 1.

FIG. 3 is a transverse cross-section of the device of FIG. 1, showing the positioning of the components at various stages.

In FIG. 1, a housing 1 constructed of plastic or other suitable rigid material has electrical pins 2 for connection to a domestic electricity mains supply. The power is used to drive a motor 3 which in turn drives an impeller 4, which is located within the housing 1 and which, when switched on, takes air in through an external grille 11 (upper, right-pointing white arrows) and blows it through the device in a manner that will be further described hereinunder. A spindle 5 from the motor 3 protrudes into the centre of the housing 1 and the impeller 4 fits tightly onto this spindle 5.

Partially surrounding the impeller is a manifold 8 having the form of a part-cylinder mounted with its longitudinal axis coincident with the spindle 5. This manifold is mounted such that it is capable of free rotation about this axis. Depending downwards from the manifold is a conduit 9 having, when viewed in back-to-front section, essentially a quarter-circular shape, such that air from the impeller 4 will first travel vertically downwards (black vertical arrows) and then horizontally out of the device (white left-pointing arrows). From the rear of the conduit protrudes a member 10 bearing a magnetic or ferromagnetic element. As the manifold 8 and therefore the conduit 9 pivot about the axis, the magnetic or ferromagnetic element comes into close proximity with a series of electromagnets 7 located in an arc, such that the activation of any one electromagnet will cause an magnetic attraction that will the conduit 9 to pivot into a position with the magnetic or ferromagnetic element in close proximity to the electromagnet.

In the embodiment of FIG. 1, the device contains three reservoirs 17 of volatile liquid 15, these passing from the reservoir via porous wicks 14. These wicks protrude into a space between the external grille 11 and the conduit 9, when the conduit is positioned behind the wick. Positioning of the conduit 9 with respect to any wick 14 is achieved by the location of three electromagnets 7 (more clearly seen in FIG. 2) such that, when a particular electromagnet is actuated, the conduit will move to that electromagnet and will be positioned to deliver an air current from the impeller 4 to the wick and out of the device into the atmosphere. The arrangement of the three wicks and the corresponding electromagnets and the conduit in three different positions is shown in FIG. 3. The reservoirs and the corresponding wicks are in the form of replaceable refills that can be removed and replaced when empty or when a different liquid is desired.

The actuating of the electromagnets is effect by adjustable electronics (not shown) within the body 1. These permit the determination of the order and the time of the dissemination of the various liquids.

Thus in operation, the device is plugged in and the electronics are actuated, with the desired order and time for each liquid set. As the electromagnets are actuated or deactivated, the conduit swings between the various wicks, directing liquid-disseminating air current over them and out of the apparatus.

The above-described embodiment is an example only and the skilled person will be able to realise many other ways of performing the invention not described herein, but lying within the scope of the invention.

1. A device adapted to disseminate into an atmosphere, in a desired sequence and timing, a plurality of active volatile liquids, comprising a plurality of individual liquid reservoirs, a dissemination element associated with each reservoir, a source of forced air current and a conduit from the source of forced air current to each dissemination element, the conduit being a single conduit that is adapted to be movable so as to supply air current to each dissemination element in a desired sequence.

2. A device according to claim 1, in which the device is a plug-in device.

3. A device according to claim 1, in which the dissemination element is selected from a porous wick and an external capillary emanator.

4. A device according to claim 1, in which the conduit is adapted to pivot in an arc.

5. A device according to claim 4, in which the conduit extends from a pivotable housing partially or completely surrounding the source of air current.

6. A device according to claim 4, in which the arc lies in a vertical plane.

7. A device according to claim 1, in which movement of the of the conduit between the plurality of dissemination elements is effected by electromagnets positioned in the vicinity of each reservoir, which electromagnets may be activated or deactivated such that the conduit moves to the desired dissemination element.

8. A device according to claim 1, in which the movement of the conduit may be programmed to any desired order of dissemination elements.

9. A method of providing sequentially in an atmosphere a plurality of volatile liquids, comprising the steps of
(a) providing each of a plurality of volatile liquids in individual reservoirs with dissemination elements extending therefrom; and
(b) providing to the dissemination elements in a desired order and timing a forced air current from a conduit, which conduit is movable so as to direct air current to the desired dissemination element.
